(19) United States
(12) Patent Application Publication
(10) Pub. No.: US 2021/0219833 A1
(43) Pub. Date: Jul. 22, 2021

(54) METHOD AND APPARATUS FOR WARMING A TIP-PORTION OF AN OPTICAL SCOPE PRIOR TO IN-VIVO USE FOR IMAGING

(71) Applicant: DWIGHT H. LENNOX, II, HIGHLANDS RANCH, CO (US)

(72) Inventor: DWIGHT H. LENNOX, II, HIGHLANDS RANCH, CO (US)

(21) Appl. No.: 16/972,180

(22) PCT Filed: Jun. 4, 2019
(86) PCT No.: PCT/US2019/035327

§ 371 Date: Dec. 4, 2020

Related U.S. Application Data
(60) Provisional application No. 62/681,198, filed on Jun. 6, 2018.

(51) Int. Cl. A61B 1/12; A61B 1/018; A61B 1/313; A61B 1/00

(52) U.S. Cl. CPC A61B 1/127; A61B 1/018; A61B 1/128; A61B 2562/0271; A61B 1/00025; A61B 1/00137; A61B 1/3132

(57) ABSTRACT

A method and apparatus for warming an optical scope prior to in-in vivo disposal wherein a thermally conductive material is received into a chamber and is then heated in order to conduct the heat to a tip-portion of the optical scope. A perimeter about tip-portion of the optical scope is sealed to prevent migration of the thermally conductive material through the orifice one the optical scope is inserted into the chamber.

### RELATED APPLICATIONS

The present application claims priority to U.S. Provisional Application Ser. No. 62/681,198, entitled “Disposable Endoscope Warming Device and Methods of Use Thereof” by Lenox, which was filed on Jun. 6, 2018 and where the text and drawings of said application are incorporated by reference into this application in their entirety.

### BACKGROUND

Inserting an endoscope, or laparoscope, that is at ambient temperature into a cavity of a warm body can cause the end of the scope to fog, thus hindering its immediate use. To prevent this problem, portable and disposable warmers are known wherein the ends of the instrument are placed in a water-based fluid contained in the warmer's cavity to warm the tip-portion of such an optical scope prior to use in surgery or another medical procedure. This warming of the tip-portion of the optical scope significantly reduces the risk of fogging the optical end of the scope. The water-based solution is typically heated by either a chemical reaction or a battery-powered electric heater.

It is not uncommon for significant amounts of the fluid to leak out of the scope insertion orifice during use. The amount of leakage depends on several factors including the orientation of the warmer. For example, such warmers are often purposefully tipped over on to their sides during use. This is done so that the long bodies of the scope can rest on the surface of a cart or table. Leakage also occurs because the diameter of the orifice compared to the diameter of the instrument inserted therein may not be perfectly matched. Leakage also occurs when the scope is not inserted in the warmer.

### BRIEF DESCRIPTION OF THE DRAWINGS

Several alternative embodiments will hereinafter be described in conjunction with the appended drawings and figures, wherein like numerals denote like elements, and in which:

FIG. 1 is a flow diagram that depicts one example method for warming an optical scope prior to in-in vivo disposal;

FIG. 2 is a flow diagram that depicts alternative example methods for receiving a thermally conductive material into a chamber;

FIG. 3 is a flow diagram that depicts alternative example methods for receiving a tip-portion of the optical scope into the chamber;

FIG. 4 is a flow diagram that depicts one alternative example method for applying heat to the thermally conductive material;

FIG. 5 is a flow diagram that depicts one alternative example method for controlling the temperature proximate to the scope;

FIG. 6 is a flow diagram that depicts one alternative example method wherein electrical power is provided to the heating element;

FIG. 7 is a flow diagram that depicts yet another alternative example method for controlling application of power to the heating element;

FIG. 8 is a flow diagram that depicts one alternative example method that promotes infection control in a surgical theater;

FIG. 9 is a flow diagram that depicts one alternative example method wherein a tip-portion of the optical scope is cleaned when it is fully inserted into a chamber intended to receive a thermally conductive material;

FIG. 10 is a flow diagram that depicts one alternative example method for impeding dissemination of fluid from the orifice when a tip-portion of the optical scope is withdrawn from the chamber;

FIG. 11 is a flow diagram that depicts one alternative example method that further provides for point-of-use decontamination of surgical instruments;

FIG. 12 is a pictorial diagram that illustrates one example embodiment of an optical scope management device;

FIG. 13 is a pictorial diagram that presents a partial cutaway view of one example embodiment of a fully assembled device for managing an optical scope;

FIG. 14 is a pictorial illustration that illustrates a cutaway view of a chamber intended to receive a thermally conductive material;

FIG. 15 presents a frontal cross-section view of a chamber and the manner in which a heating element and temperature sensor are integrated therewith;

FIG. 16 is a pictorial diagram that illustrates one example embodiment of a barrier-strip power gate;

FIG. 17 is a pictorial diagram that illustrates one example alternative embodiment of a battery cover that includes breakaway retention elements;

FIG. 18 is a pictorial diagram that illustrates the mechanics of the breakaway retention elements used to retain the battery cover; and

FIGS. 19 and 20 are pictorial diagrams that illustrate the structure and operation of an eyelid seal.

### DETAILED DESCRIPTION

In the interest of clarity, several example alternative methods are described in plain language. Such plain language descriptions of the various steps included in a particular method allow for easier comprehension and a more fluid description of a claimed method and its application. Accordingly, specific method steps are identified by the term “step” followed by a numeric reference to a flow diagram presented in the figures, e.g. (step 5). All such method “steps” are intended to be included in an open-ended enumeration of steps included in a particular claimed method. For example, the phrase “according to this example method, the item is processed using A” is to be given the meaning of “the present method includes step A, which is used to process the item”. All variations of such natural language descriptions of method steps are to be afforded this same open-ended enumeration of a step included in a particular claimed method.

Unless specifically taught to the contrary, method steps are interchangeable and specific sequences may be varied according to various alternatives contemplated. Accordingly, the claims are to be construed within such structure. Further, unless specifically taught to the contrary, method steps that include the phrase “. . . comprises at least one or more of A, B, and/or C . . . ” means that the method step is to include every combination and permutation of the enumerated elements such as “only A”, “only B”, “only C”, “A and B, but not C”, “B and C, but not A”, “A and C, but not B”, and “A and B and C”. This same claim structure is also intended to be open-ended and any such combination of the enumerated elements together with a non-enumerated element, e.g. “A and D, but not B and not C”, is to fall within the scope of the claim. Given the open-ended intent of this claim language, the addition of a second element, including an additional of an enumerated element such as “2 of A”, is to be included in the scope of such claim. This same intended claim structure is also applicable to apparatus and system claims.

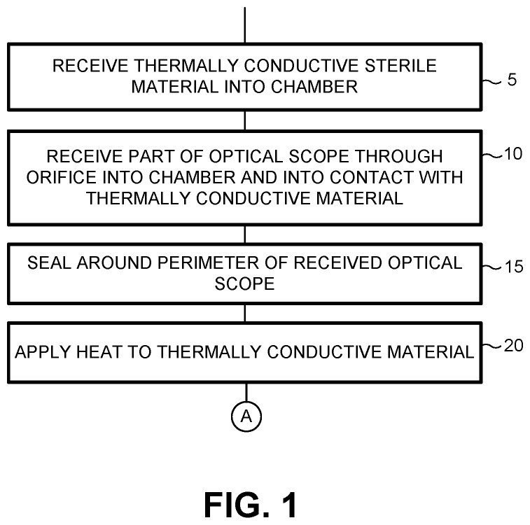

FIG. 1 is a flow diagram that depicts one example method for warming an optical scope prior to in-in vivo disposal. This example method includes a step that provides for receiving a thermally conductive material into a chamber (step 5). Once the thermally conductive material is received in the chamber, an additional step is included for receiving part of an optical scope through an orifice, wherein the orifice provides access to the chamber (step 10). It should be appreciated that, according to this example method, this step is accomplished once a tip-portion of the optical scope makes contact with the thermally conductive material disposed within the chamber.

An additional included step provides for sealing around the perimeter of the tip portion of the optical scope (step 15). It should be appreciated that sealing around the perimeter of the tip-portion of the optical scope substantially prevents the loss of thermally conductive material from the chamber through the orifice.

This example method further includes a step for applying heat to the thermally conductive material (step 20). Accordingly, the heat is transferred to the thermally conductive material and thus transmitted to the tip-portion of the optical scope.

This example method, according to one illustrative use case, is used in a surgical theater in order to raise the temperature of a tip-portion of an optical scope so that said tip-portion of the optical scope does not fog up once it is disposed in a patient's body.

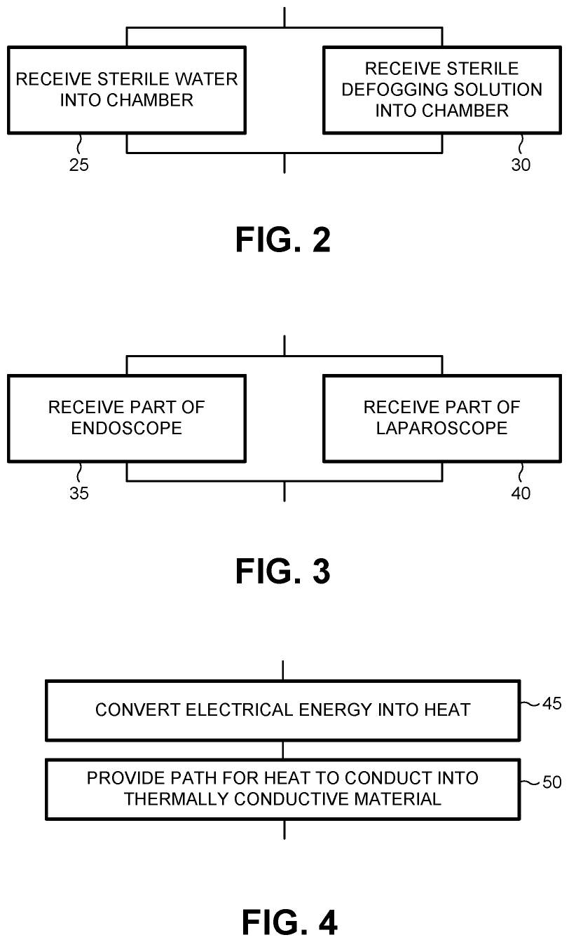

FIG. 2 is a flow diagram that depicts alternative example methods for receiving a thermally conductive material into a chamber. It should be appreciated that, according to one alternative example method, an included step provides for receiving sterile water into the chamber (step 25). It should be appreciated that sterile water is a relatively good conductor of heat. Accordingly, once heat is applied to the sterile water, the heat is then transferred to the tip-portion of an optical scope.

In one alternative example method, a sterile defogging solution is received into the chamber (step 30) in an included step. A sterile defogging solution includes chemicals that help to deter fogging of an optical scope beyond merely raising the temperature of the tip-portion of the optical scope so as to substantially match the temperature of a body. It should be appreciated that the claims appended hereto are not intended to be limited to applications where an optical scope is used for a human patient.

FIG. 3 is a flow diagram that depicts alternative example methods for receiving a tip-portion of the optical scope into the chamber. It should also be appreciated that, according to various illustrative use cases, the optical scope includes at least one or more of an endoscope and/or a laparoscope. It should likewise be appreciated that the present method is applicable when using various types of optical scopes and the claims appended hereto are not intended to be limited to any particular optical scope herein described or otherwise enumerated for the purpose of example.

This being said, one alternative example method includes a step that provides for receiving part of an endoscope (step 35) into the chamber. In yet another alternative example method, a step is included for receiving a part of a laparoscope into the chamber (step 40). Again, it is important to note that the method herein described is applicable for warming a wide range of optical scopes and the claims appended hereto are not intended to be limited to any particular type of optical scope herein enumerated.

FIG. 4 is a flow diagram that depicts one alternative example method for applying heat to the thermally conductive material. In this alternative example method, an included step provides for converting electrical energy into heat (step 45) and then providing a path for the heat to be conducted into the thermally conductive material (step 50). It should be appreciated that, according to one illustrative use case, a chamber has disposed thereon an electrically-driven heating element and a thermally conductive path disposed between the heating element and the thermally conductive material.

It should likewise be appreciated that, according to various alternative example embodiments that implement the present method, the chamber itself is made of a thermally isolating material so that, once heat is introduced into the chamber, the heat is retained and not allowed to propagate outward through the walls of the chamber itself.

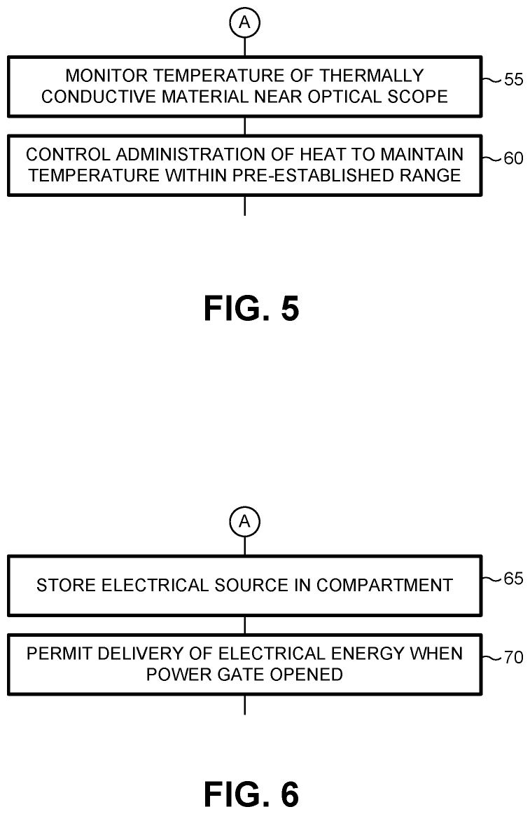

FIG. 5 is a flow diagram that depicts one alternative example method for controlling the temperature proximate to the scope. It should be appreciated that, according to various alternative example methods, heat is applied to the thermally conductive material in a manner such that the heat is communicated to a tip-portion of the scope. Accordingly, one alternative example method further includes a step for monitoring the temperature of the thermally conductive material proximate, or near to the optical scope (step 55). According to one illustrative use case, this method is utilized in a device where a temperature sensor is disposed proximate to the optical scope. In yet another alternative example method, the temperature of the thermally conductive material is monitored proximate to a tip-portion of the optical scope.

As such, the amount of heat applied to the thermally conductive material is regulated in order to maintain the temperature of the thermally conductive material, as monitored, within a particular pre-established range (step 60) as provided for in a further included step. It should be appreciated that, according to one illustrative use case, this variation of the present method is applied so that the temperature proximate to the optical scope, as monitored, is maintained in a range conducive to reducing fogging of the tip-portion of the optical scope when inserted into a body. Again, according to some illustrative use cases of the present method and variations thereof, the method is used when warming a tip-portion of an optical scope for use in a human body. This, though, is not intended to limit the scope of the claims appended hereto.

It should likewise be appreciated that, according to yet another illustrative use case, the amount of heat is regulated in order to maintain the monitored temperature of the thermally conductive material in a range centered about normal body temperature range. For example, when utilizing the present method with a human patient, the temperature range is centered about 98.6° F. This alternative method is also not intended to limit the scope of the claims appended hereto.

FIG. 6 is a flow diagram that depicts one alternative example method wherein electrical power is provided to the heating element. According to this example alternative method, further steps include providing an electrical source, wherein the electrical source is stored in a compartment (step 65); and allowing power to flow to the heating element when a power gate is opened (step 70). According to various illustrative use cases, the power gate is implemented in a device that practices the present method and variations thereof by means of an electrical switch.

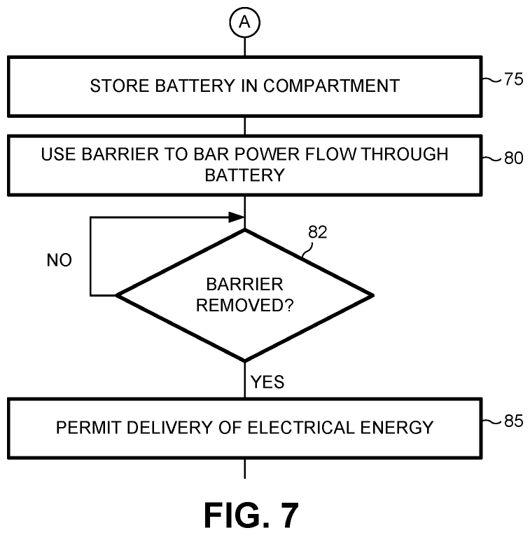

FIG. 7 is a flow diagram that depicts yet another alternative example method for controlling application of power to the heating element. According to one illustrative use case, this alternative example method is utilized in an apparatus where electrical power is provided from a battery, said battery being disposed in a battery holder (a.k.a. a battery compartment) included in a device that includes the chamber for receiving the thermally conductive material. According to yet another illustrative use case, an insulating strip is disposed between a terminal included on a battery and an electrical contact that is included in the battery holder. Accordingly, this illustrative use case provides that power is allowed to flow from the battery to the electrical contact included in the battery holder when the insulating strip is removed.

As such, this alternative example method further includes a step for storing a battery in a battery compartment (step 75), using a barrier to prevent power flow through the battery (step 80), and permitting power to flow from the battery (step 85) when the barrier is removed (step 82).

It should be appreciated that, according to yet another illustrative use case, a device that implements the present example method and variations thereof is provided in a “ready to use” manner. In this illustrative use case, batteries are included with the device and power is allowed to flow when the insulating strip is removed. Before the insulating strip is removed, the insulating strip prevents the terminal included on the battery from making electrical contact with the electrical contact included in the battery holder.

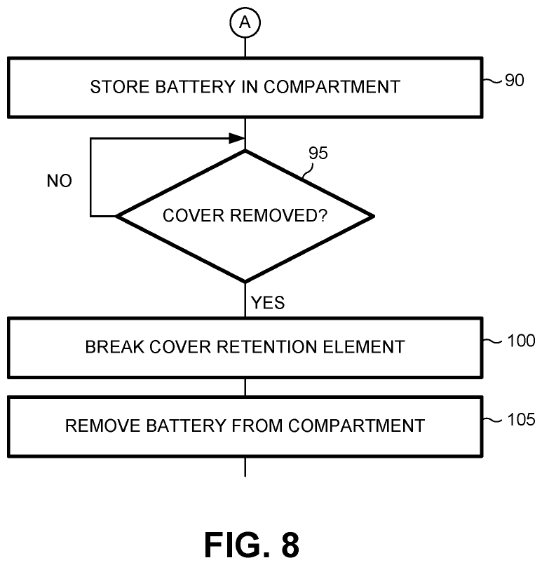

FIG. 8 is a flow diagram that depicts one alternative example method that promotes infection control in a surgical theater. This alternative example method further includes a step for storing a battery in a battery compartment (step 90); breaking a cover retention element included on a cover intended to cover the battery compartment (step 100) when the cover is removed (step 95); and removing the battery from the compartment (step 105).

It should be appreciated that this alternative example method promotes infection control by promoting single use of a device that implements the example method and variations thereof. It should further be appreciated that, at least according to one illustrative use case, a “ready to use” device includes batteries that are enabled when the device is used in a surgical theater. It should likewise be appreciated that, in order to further promote infection control, the device itself is discarded, and likely incinerated. Because it is not environmentally unconscionable to incinerate batteries, it becomes incumbent upon the surgical staff to remove the batteries from a device prior to disposal.

According to one alternative example embodiment of a device that implements the present method and variations thereof, a battery compartment is covered by a battery compartment cover which is intended to be used only one time. This is discussed further, infra.

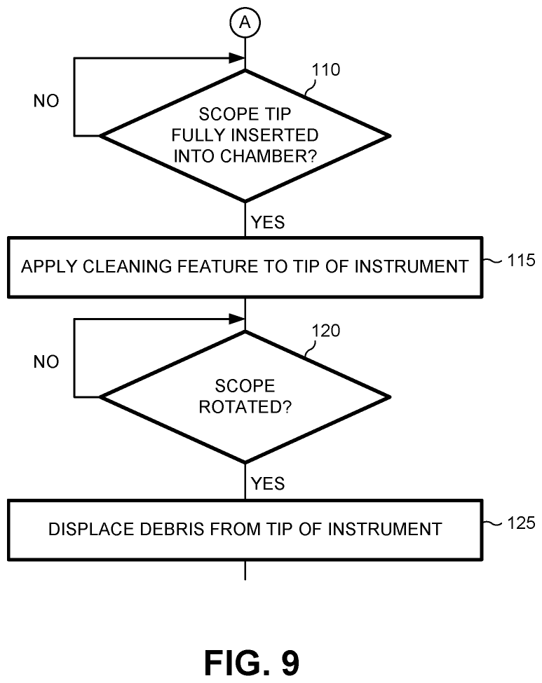

FIG. 9 is a flow diagram that depicts one alternative example method wherein a tip-portion of the optical scope is cleaned when it is fully inserted into a chamber intended to receive a thermally conductive material. In this alternative example method, additional steps are included for applying a cleaning feature to the tip of the optical scope (step 115) when a tip-portion is fully inserted into the chamber (step 110). Yet an additional included step is provided for displacing debris from the tip-portion of the optical scope (step 125) when the optical-scope is rotated while the tip-portion is fully inserted into the chamber that receives thermally conductive material.

According to various illustrative use cases, the thermally conductive material comprises a liquid that is substantially efficacious in promoting cleaning of a tip-portion of an optical scope. Accordingly, one illustrative use case provides for a cleaning feature within the chamber such that the cleaning feature makes contact with the tip-portion of the optical scope when the optical scope is fully inserted into the chamber.

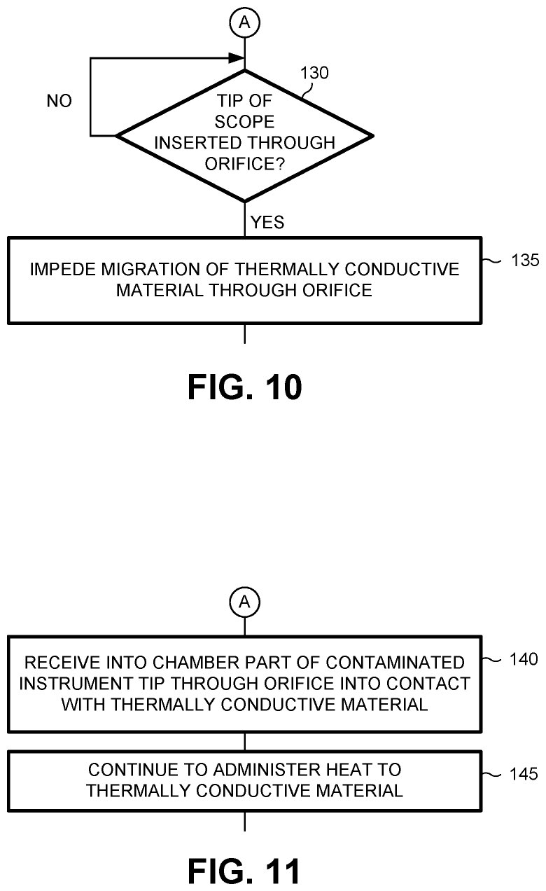

FIG. 10 is a flow diagram that depicts one alternative example method for impeding dissemination of fluid from the orifice when a tip-portion of the optical scope is withdrawn from the chamber. It should be appreciated that, once the optical scope is withdrawn from the chamber, it is important to contain the thermally conductive material within the chamber. This substantially precludes contamination of the surgical theater environment where devices that implement the present method and variations thereof are likely to be used.

As such, this alternative example method further includes a step for substantially preventing the migration of the thermally conductive material through the orifice (step 135) when the tip-portion of the optical scope is not inserted through the orifice into the chamber (step 130). According to one illustrative use case, this alternative example method is embodied in a device that includes an eye-lid valve disposed across the orifice. It should be appreciated that a device the implements the teachings of the present method is first provided to a user without an optical scope being inserted into the orifice. Hence, these additional method steps are applicable before the tip-portion is inserted into the chamber containing the thermally conductive material.

FIG. 11 is a flow diagram that depicts one alternative example method that further provides for point-of-use decontamination of surgical instruments. It should be appreciated that, in order to more effectively sterilize surgical instruments after use in the surgical theater, it is advantageous to maintain the tip-portion of any instrument moist. Accordingly, this alternative example method provides an included additional step for receiving into the chamber part of a contaminated surgical instrument into the chamber through the orifice in order to make contact with the thermally conductive material contained therein (step 140). According to yet another alternative method, an additional included step provides for the continued application of heat to the thermally conductive material (step 145). Hence, not only does this alternative method provide for keeping the tips of a surgical instrument moist, this alternative method provides for keeping the tip of the surgical instrument warm, relative to ambient temperatures, which may be advantageous to removal of biological contamination during a subsequent cleaning and sterilization process.

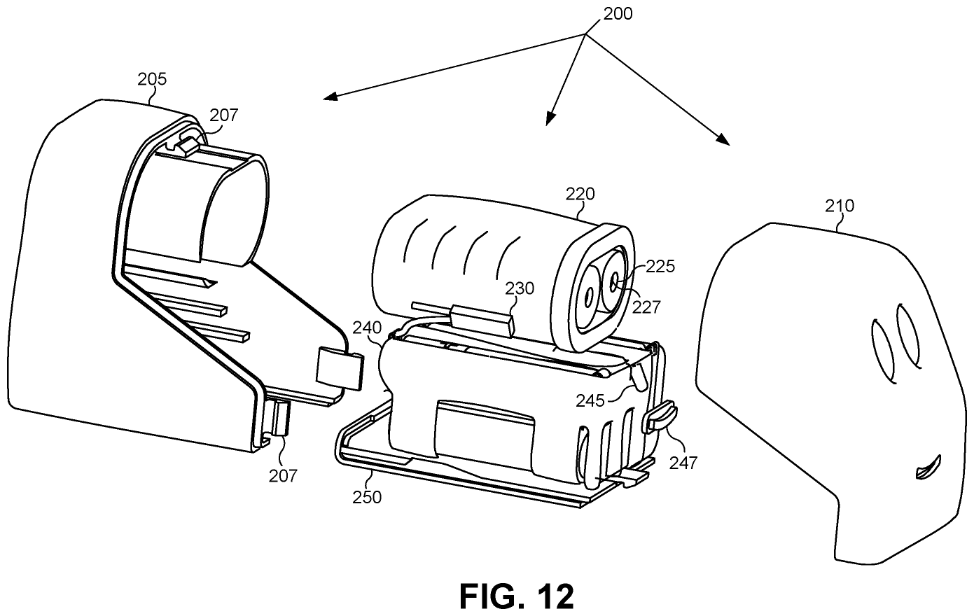

FIG. 12 is a pictorial diagram that illustrates one example embodiment of an optical scope management device. According to this alternative example embodiment, a device for managing an optical scope 200, which is used for in vivo imaging, comprises a chamber 220, an orifice 225, a sealing device 227 disposed across the orifice 225, and a heating element 230. According to one alternative example embodiment, the optical scope management device 200 further includes an indicator 245 for indicating when the heating element 230 is engaged.

It should be appreciated that the chamber 220 is intended to receive a thermally conductive material. In operation, the orifice 225 is used for receiving into the chamber 220 a tip-portion of an optical scope. The sealing device 227 disposed across the orifice 225 is fashioned to substantially prevent migration of a thermally conductive material received into the chamber 220 when an optical scope is not inserted through the orifice 225. The heating element 230 is used for warming thermally conductive material received into the chamber 220.

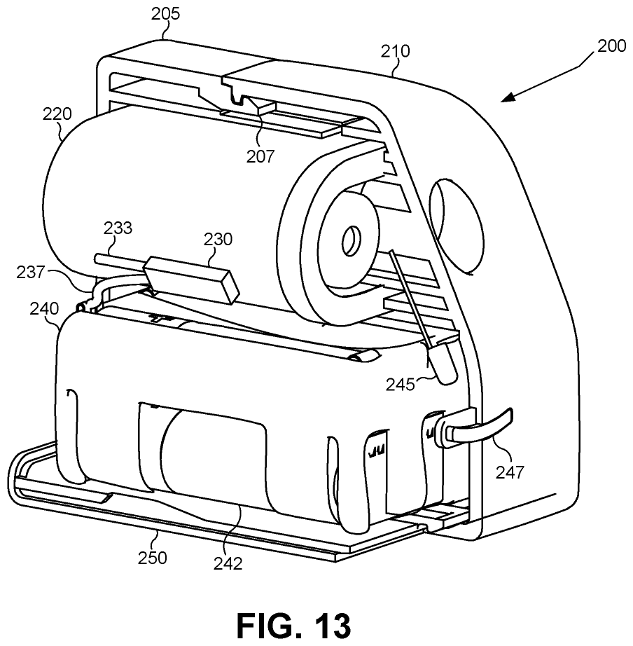

FIG. 13 is a pictorial diagram that presents a partial cutaway view of one example embodiment of a fully assembled device for managing an optical scope. According to yet another alternative example embodiment, the device for managing an optical scope 200 also comprises a rear enclosure 205, and a front enclosure 210. The rear enclosure 205 and the front enclosure 210 are used to encapsulate the chamber 220 and the heating element 230. It should be appreciated that, as illustrated in the figure, the rear in enclosure 205 and the front enclosure 210 are held together by means of one or more clasps 207. It should be appreciated that, according to this example embodiment, the clasps 207 allow the rear enclosure 205 and the front enclosure 210 to “snap together”. The clasps 207 of this alternative embodiment are fashioned to impede disassembly of a fully assembled device 200.

FIG. 13 further illustrates that, according to yet another alternative example embodiment, a device for managing an optical scope 200 further includes a battery receptacle 240 for receiving a battery 242. This alternative example embodiment also includes a power gating device 247. In this alternative example embodiment, the power gating device 247 comprises an electrical switch that also allows for illumination by means of an indicator 245. It should be appreciated that, in accordance with common practice, when the power gating device enables power from the battery 242 to reach the heating element 230, power is also delivered to the indicator 245. It should be appreciated that, according to this alternative example embodiment, electrical power from the battery 242 is conveyed through the power gating device 247 to the heating element 230 by means of an electrical connection 237. In yet another alternative example embodiment, the opening to the battery receptacle 240 is covered by a battery cover 250.

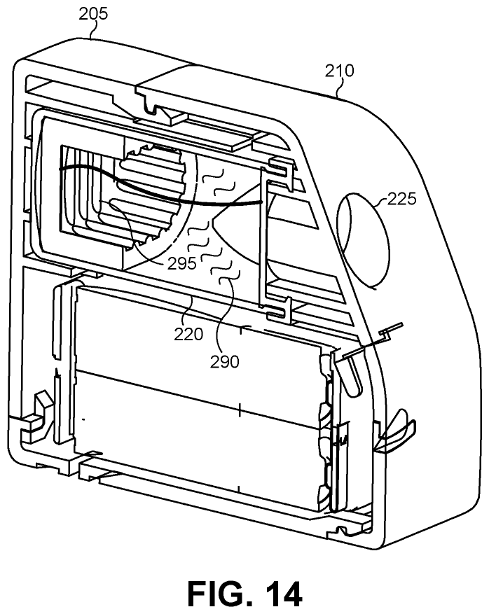

FIG. 14 is a pictorial illustration that illustrates a cutaway view of a chamber intended to receive a thermally conductive material. It should be appreciated that, according to one alternative example embodiment, the chamber 220 is pre-filled with a thermally conductive material 290. According to various alternative example embodiments, the chamber 220 is pre-filled with at least one or more of sterile water and/or sterile defogging solution.

FIG. 14 further illustrates that, according to one alternative example embodiment, a cleaning surface 295 is included in the device and is disposed on an internal surface of the chamber 220. In this alternative example embodiment, the cleaning surface 295 is set back away from the orifice 225, substantially at a rear-portion of the chamber 220. Accordingly, a tip-portion of an optical scope will make contact with the cleaning surface 295 when the optical scope is moved further into the chamber 220.

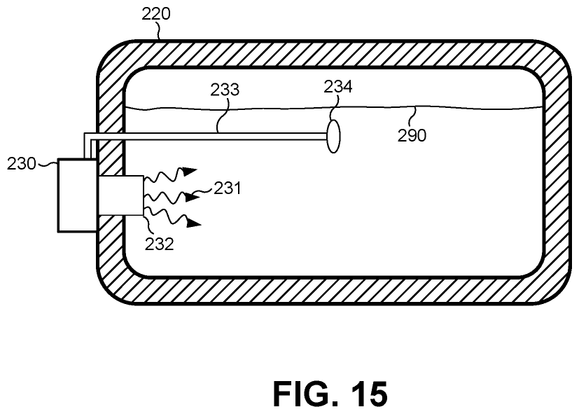

FIG. 15 presents a frontal cross-section view of a chamber and the manner in which a heating element and temperature sensor are integrated therewith. It should be appreciated that, according to one alternative example embodiment, the chamber 220 comprises walls that are resistant to the flow of heat across their boundaries. Once heat is introduced into the chamber 220 by the heating element 230, the insulating walls enable the chamber 222 to further retain the heat, rather than allowing the heat to escape into the ambient environment.

Accordingly, this alternative example embodiment of a chamber 220 receives a heating core 232, which is included as a portion of the heating element 230, through the insulating walls into a portion of the chamber that is intended to contain thermally conductive material 290. The heating core portion 232 of the heating element 230 then introduces heat 231 into the thermally conductive material 290.

FIG. 15 also illustrates that, according to one alternative embodiment, the device includes a thermal sensor 234. In yet another alternative embodiment, the thermal sensor is disposed in a portion of the chamber that is proximate to where an optical scope is inserted into the thermally conductive material 290. Hence, this alternative embodiment provides a support member 233 that extends into the chamber so as to provide a mounting structure for the thermal sensor 234.

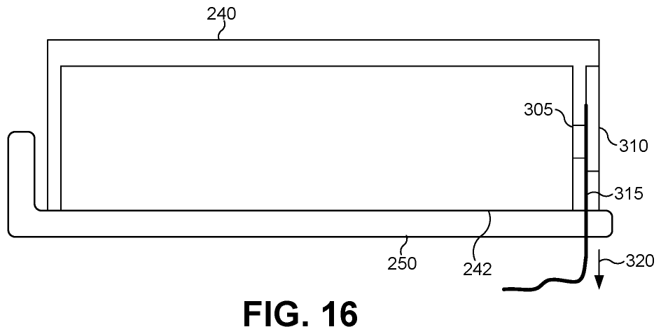

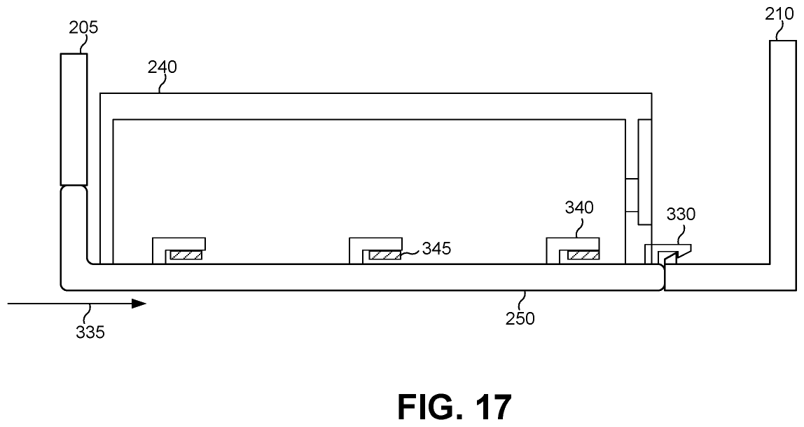

FIG. 16 is a pictorial diagram that illustrates one example embodiment of a barrier-strip power gate. As heretofore described, one alternative example method relies upon a power barrier in order to provide for a single use application of power from the battery 242 to the heating element 230. In one alternative example embodiment, a barrier-strip 315 is initially disposed between a contact 305 included in the battery 242 and a contact 310 which is included in the battery receptacle 240. In operation, a user will pull down 320 on the barrier-strip 315. As such, when the barrier-strip 315 is removed the battery contact 305 comes into electrical contact with the electrical contact 310 included in the battery receptacle 240. Thus, electrical power is allowed to flow from the battery 242 to the electrical contact 310 included in the battery receptacle 240 when the barrier-strip is removed.

FIG. 17 is a pictorial diagram that illustrates one example alternative embodiment of a battery cover that includes breakaway retention elements. According to one alternative example embodiment, the battery cover 250 includes one or more retention elements 340 that engage with a corresponding retention feature 345. It should be appreciated that the corresponding retention features 345, according to this alternative example embodiment, are included and made part of the rear enclosure 205.

According to this alternative example embodiment, the battery cover 250 is placed up into an opening directly opposite the battery receptacle 240. The battery receptacle 250 slides forward 335 in order to engage with the corresponding retention features 345, which are included in the rear enclosure 205. In this example embodiment, the battery cover 250 also includes a clasp 330 that engages with a corresponding clasp included in the front enclosure 210. As such, as the battery cover 250 slides forward and the clasp 330 engages with his counterpart, the battery cover “snaps into place”. The clasp 330 substantially precludes the battery cover 250 from sliding backward once said clasp 330 is engaged with his counterpart. It should likewise be appreciated that the breakaway retention elements 340 substantially prevent the battery cover 250 from falling away from the battery receptacle 240.

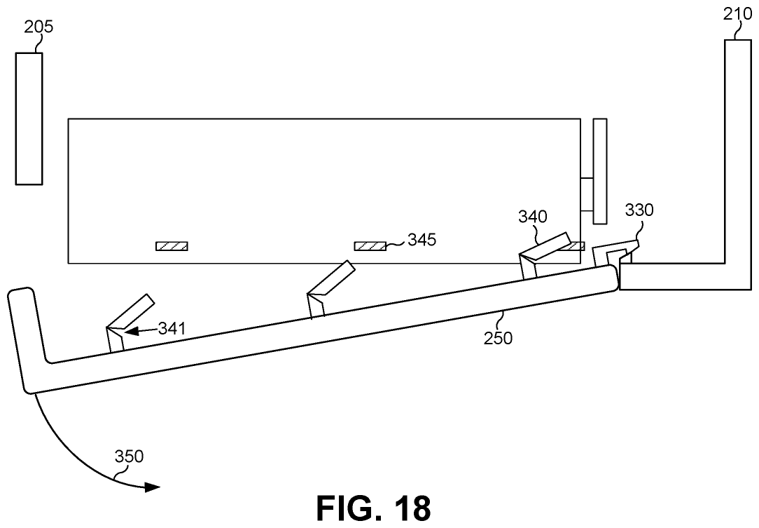

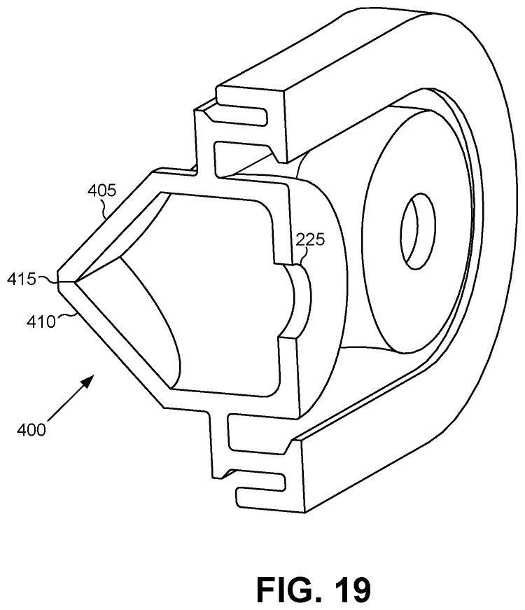

FIG. 18 is a pictorial diagram that illustrates the mechanics of the breakaway retention elements used to retain the battery cover. Because the clasp 330 included in the battery cover 250 prevents the battery cover from sliding back relative to the retention features 345 included in the rear enclosure 205, removal of the battery cover 250 requires that it be pulled away from the rear enclosure 205. The clasp 330, in this alternative example embodiment, also prevents the front portion of the battery cover 250 from being pulled away from the front enclosure 210. The rear of the battery cover 250 is then pulled down in an arc 350. As the battery cover 250 is pulled down in this fashion, the break-away retention elements 340 do just that, they break away from the corresponding retention features 345 included in the rear enclosure 205.

It should be appreciated that the retention features 340 included in the battery cover 250 are purposefully manufactured with a breakaway feature 341 so that breakaway occurs when the rear of the battery cover 250 is pulled away from the rear enclosure 205. According to this alternative example embodiment, the breakaway feature 340 comprises a weekend right-angle construction that breaks when the retention element 340 pulls away from the corresponding retention feature 345 included in the rear enclosure 205.

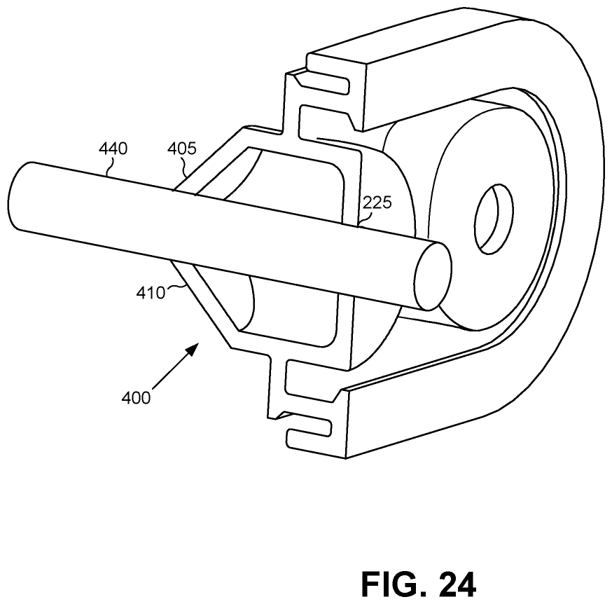

FIGS. 19 and 20 are pictorial diagrams that illustrate the structure and operation of an eyelid seal. According to one alternative example embodiment, an eyelid seal 400 includes two flaps 405, 410. When there is no optical scope inserted through the orifice 225, the two flaps 405 and 410 make contact with each other creating an interface 415 between the two flaps. This interface 415 forms a seal that substantially prevents fluid from moving across the eyelid seal 400. It should be appreciated that the eyelid seal 400 is constructed of a resilient and pliable material

In this example embodiment, the eyelid seal 400 is integrated with an orifice 225 and is constructed of a resilient and pliable material. The diameter of the orifice 225 is selected to be slightly smaller than the outside diameter of an optical scope.

FIG. 20 illustrates that, according to this alternative example embodiment, the orifice 225, by virtue of the resilient and pliable material it is constructed of, forms a seal about the perimeter of an optical scope 440 when the optical scope 440 is inserted through the orifice 225. As the optical scope 440 is pushed further through the orifice 225, it pushes aside the two flaps 405, 410 included in the eyelid seal 400. It should be appreciated that, because the eyelid seal 400 includes an essentially linear interface 415 between the two flaps 405, 410, a perfect seal is not formed about the perimeter of the optical scope 440. Hence, some limited amount of thermally conductive material will enter into the void between the flaps 405, 410 and the orifice 225, said orifice creating a seal about the perimeter of the optical scope 440. When the optical scope 440 is extracted from the chamber 220, the two flaps 405, 410 included in the eyelid seal 400 return to their original position, as shown in FIG. 19. The two flaps 405, 410 return to their original position because of the resiliency of the material from which they are formed.

While the present method and apparatus has been described in terms of several alternative and exemplary embodiments, it is contemplated that alternatives, modifications, permutations, and equivalents thereof will become apparent to those skilled in the art upon a reading of the specification and study of the drawings. It is therefore intended that the true spirit and scope of the claims appended hereto include all such alternatives, modifications, permutations, and equivalents.

What is claimed is:

1. A method for warming an optical scope prior to in-vivo disposal comprising:
receiving a thermally conductive material into a chamber, said thermally conductive material being substantially sterile;
receiving through an orifice a tip portion of the optical scope into the chamber so as to make contact with the thermally conductive material;
sealing around the perimeter of the tip portion of the optical scope; and
applying heat to the thermally conductive material.

2. The method of claim 1 wherein receiving a thermally conductive material into a chamber comprises:
receiving into the chamber at least one or more of sterile water and/or sterile defogging solution.

3. The method of claim 1 wherein receiving a tip portion of the optical scope comprises:
receiving at least one or more of a tip portion of an endoscope and/or a laparoscope.

4. The method of claim 1 wherein applying heat to the thermally conductive material comprises:
converting electrical energy into heat; and
providing a thermal path for the heat to conduct into the thermally conductive material.

5. The method of claim 1 further comprising:
monitoring the temperature of the thermally conductive material proximate to the tip portion of the optical scope; and
controlling the application of heat in order to maintain a temperature substantially within a pre-established range.

6. The method of claim 1 further comprising:
storing an electrical source in a compartment; and
enabling delivery of electrical energy when a power gate is opened.

7. The method of claim 1 further comprising:
storing a battery in a compartment;
preventing power flow through the battery by means of a barrier; and
enabling delivery of electrical energy when the barrier is removed.

8. The method of claim 1 further comprising:
storing a battery in a compartment;
breaking a cover retention element when a cover is removed from the battery compartment; and
removing the battery from the compartment.

9. The method of claim 1 further comprising:
applying a cleaning feature to a tip of the optical scope when the tip portion is fully inserted into the chamber; and
dislodging debris from the tip of the optical scope when the optical scope is rotated.

10. The method of claim 1 further comprising:
substantially preventing the thermally conductive material from migrating through the orifice when the tip portion of the optical scope is withdrawn from the chamber.

11. The method of claim 1 further comprising:
receiving through the orifice a tip portion of a contaminated surgical instrument into the chamber so as to make contact with the thermally conductive material.

12. The method of claim 11 further comprising continuing to apply heat to the thermally conductive material.

13. An apparatus for managing an optical scope for in vivo imaging comprising:
chamber for receiving a thermally conductive material;
orifice for receiving into the chamber a tip of the optical scope;
sealing device disposed for substantially sealing the orifice when an optical scope is not inserted into said orifice; and
heating element for warming the thermally conductive material.

14. The apparatus of claim 13 wherein the chamber is pre-filled with at least one or more of sterile water and/or sterile defogging solution.

15. The apparatus of claim 13 further comprising a heating controller that includes a temperature sensor disposed to make contact with a thermally conductive material disposed in the chamber and that applies power to the heating element to maintain the temperature proximate to the chamber within a pre-established range.

16. The apparatus of claim 13 further comprising:
battery receptacle for receiving a battery; and
power gating device that, when actuated, causes power to be applied from battery terminals included in the battery receptacle to the heating device.

17. The apparatus of claim 13 further comprising:
battery receptacle for receiving a battery; and
single-use power gating device that, when actuated, causes power to be applied from battery terminals included in the battery receptacle to the heating device.

18. The apparatus of claim 13 further comprising:
battery receptacle for receiving a battery; and
cover plate for substantially preventing removal of a battery from the battery receptacle and wherein the cover plate includes a retention element that maintains the position of cover plate relative to the battery receptacle and wherein the retention element breaks when the cover plate is removed from the battery receptacle.

19. The apparatus of claim 13 further comprising:
cleaning surface disposed on an internal surface of the chamber and situated to be contacted by a tip of an optical scope when the optical scope is moved further into the chamber.

20. The apparatus of claim 13 wherein the sealing device includes a flexible eyelid-seal that includes two flaps positioned in opposition to each other wherein one side of one flap is maintained in contact with one side of the second flap by resilience of the eyelid-seal material.

21. The apparatus of claim 13 wherein the sealing device includes a flexible flap system that includes one or more flaps positioned to create a seal when an optical scope is not inserted into the chamber.
